(19) United States
(12) Patent Application Publication
(10) Pub. No.: US 2007/0105754 A1
(43) Pub. Date: May 10, 2007

(54) Pharmaceutical composition to prevent and treat epithelial wounds, immunomodulating pharmaceutical composition, pharmaceutical composition, insecticide, insecticide composition insecticide use of lectin KM+ to treat cicatrizations, use of lectin KM+ to prepare immunomodulating medicament, use of lectin KM+ to prepare anti-bacterial medicament, use lectin KM+ to prpare anti-viral medicament, use of lectin KM+ to prepare anti-parasite medicament, use of lectin KM+ to prepare anti-fungal medicament, use of lectin KM+ to prepare anti-parasite medicament expression method, DNA vector, recombinant

(76) Inventors: Luis Lamberti Pinto Da Silva, Cubotso (BR); Ademilson Panunto Castelo, Ribeirao Preto (BR); Maria Helena De Souza Goldman, Ribeirao Preto (BR); Maria Cristina Roque Antunes Barreira, Ribeirao Preto (BR); Ricardo Santos De Oliveira, Ribeirao Preto (BR); Marcelo Dias Baruffi, Ribeirao Preto (BR); Jeanne Blanco De Molfetta Machado, Piracicaba (BR)

Correspondence Address: Thomas A. O'Rourke;Bodner & O'Rourke, 425 Broadhollow Road, Melville, NY, 11747, US

(21) Appl. No.: 11/271,509

(22) Filed: Nov. 10, 2005

(51) Int. Cl. A61K 38/16; A01N 65/00; C07H 21/04; C12P 21/06; C12N 1/21; C07K 14/42; C12N 15/74

(52) U.S. Cl. 514/8; 530/396; 435/252.3; 435/472; 435/69.1; 536/23.5

(57) ABSTRACT

The invention deals with a pharmaceutical composition comprising lectin KM+ to prevent and heal epithelial wounds. The invention also comprises the use of lectin KM+, obtained from the plant (Artocarpus integrifolia) or recombinant (expression heterologue) to prepare medicaments.

### FIELD OF THE INVENTION

The invention deals with a pharmaceutical composition comprising lectin KM+ to prevent and heal epithelial wounds. The invention also comprises the use of lectin KM+, obtained from the plant (Artocarpus integrifolia) or recombinant (expression heterologue) to prepare medicaments.

### BACKGROUND OF THE INVENTION

Lectins are proteins, or glycoproteins of distribution located in the nature, that if the sugars bind selectively and reversibly. Two lectins had been isolated of seeds of Artocarpus integrifolia (jaca): jacaline and KM+, also called artocarpine. Lectin KM+ is obtained from the saline extract of seeds of Artocarpus integrifolia. The D-manose is leagued selectively being minority in the saline extract of the seeds (0.5% of total proteins), contrasting with the high concentration reached for jacaline (30% of total proteins), ligant lectin of D-galactose, contained in the same saline extract. Related studies to lectin KM+ mention the standardization to it of method for its purification, made from the saline extract of the seeds for chromatography of affinity in columns of immobilized sugar in two stages. In the first extract it is depleted of jacaline for adsortion to the immobilized D-galactose. To follow the depleted extract it with solution of D-manose is cromatographated in column of D-manose, eluded itself on lectin. The method provides homogeneous preparations of the lectin that had been used in the determination of its primary structure and in the attempt of crystal attainment for determination of the structure 3-D of the protein. The crystallization process is made difficult due to the small amounts of lectin provided by the purification method, reason for which the three-dimensional structure is proposal for molecular modeling.

The attraction of human neutrophils was the first described biological property of KM+, was detected by Saint-of-Oliveira et al. in 1994, through carried through assays in vitro and in alive. The molecular analysis of the phenomenon unchained for the stimulation of neutrophils for the lectin, led to the identification of that KM+ recognizes glicanes of cellular surface and that its linking to such glicanes active the cells, inducing its migration for small farms of bigger concentration of lectin.

The movement of neutrophil induced for KM+ if gives for haptotaxy, made possible for the fact of the lectin to be tetravalent, the domains of recognition of KM+ sugar form a bridge enter the surface of neutrophil and the laminine glycoprotein of the extracellular matrix. The ideal conditions for an adjusted cellular movement are established thus. This model of interactions revealed applicable for the induction of induced cellular movement for endogenous lectins, as galectin-3 and MNCF, as Dias-Baruffi et al, 1999.

The observation that the interaction of KM+ with glicanes of neutrofilic surface is capable to unchain dependent answers of cellular signaling, led to the assumption that the lectin could activate other immunitary cells and of this way to correspond to adjuvant an interesting one in immunization processes. It was verified initially that cells of the peritoneal socket of mice produced a great amount of interferon gamma under stimulation of KM+, and that such production was indirectly induced for the secretated I1-12 in high concentrations for the linking of KM+ to the surface of the macrophages, contained in the preparation of peritoneal cells. As such profile of citocines it is compatible with the establishment of imunitary answers of standard TH1, was opted to investigating the benefit eventually brought by the use of KM+ as adjuvant in the immunization against a sensible patogen answers TH1. The chosen model was of infection for Leishmania the major of mice of highly susceptible race to the patogen (BALB/c).

Previously to the infection the animals had been inoculated with a soluble antigen preparation of Leishmania (SLA) associate, or not it KM+. The study with L. major it showed that the animals that had received KM+ had substituted the standard of susceptibility for a standard of resistance to the parasite, as well as had inverted standard TH2 of citocines produced for a profile TH1. Thus the KM+ administration can intervene in an imunitarie reply of form to become it efficient against certain types of patogens. A time that this independent effect of the concomitant administration of the parasitic antigen, was transferred to consider it that KM+ is endowed with imunomodulator property, instead of adjuvant. As the rigorous purification of KM+ provided low income and that the excellent biological properties of KM+ imposed biotechnological investments that-made possible its application in ample scale, it was opted to proceeding the clonage and characterization from the DCNcA that codifies the lectin, aiming at to arrive the recombinant form of this lectin, through its heterolog expression. Such availability of recombinant lectin makes possible its application as therapeutical agent in injuries for burnings, as well as cicatrizant of injuries of diverse nature, as well as other pharmaceutical and/or biotechnological uses.

A library of DCNcA from extracted total RNA of jaca seeds was constructed (Artocarpus integrifolia). The DCNcAs had been clonated of directional form in the vector pSPORT-P of the Life Technologies. Such library, with approximately 13000 clones was organized in 136 plates of 96 wells (A01 the H12), contends supply in glicerol of each one of clones.

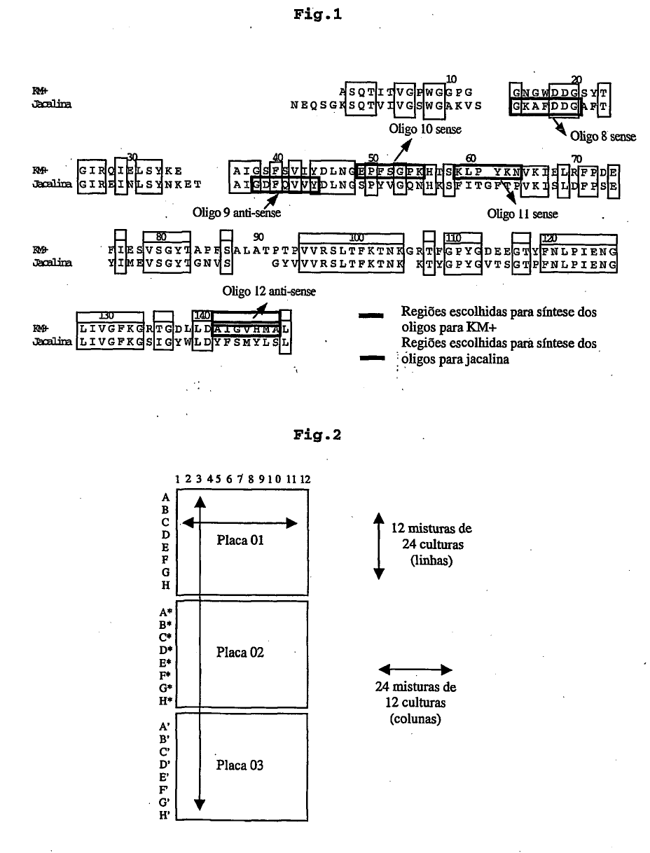

Based in the amino acid sequence of described lectin KM+ for Rose et al, (1999), and in the alignment of amino acid sequences of jacaline lectins and KM+ (FIG. 1, SEQ. ID NO:1), had been defined regions of KM+, distinct of jacaline, for which they had been drawn oligonucleotides depraved (oligo 10, oligo 11 and oligo 12, FIG. 2).

The election of the library of DCNcA of jaca seeds was carried through by PCR of matrix, from the mixture commanded of clones of DCNcA (mixture of the 136 clones in the A01 position, of the 136 clones of the A02 position and thus successively, until the 136 mixture of clones of the H12 position. The first plate I contend 96 136 mixtures of clones in each well, was used as source of DNA for the amplification for PCR. The first amplification for PCR was made with oligonucleotides SP6 (for the present sequence in the vector pSPORT-P) and oligol2. Of the result of the 96 amplifications, analyzed for eletroforese in gel of agarose, 28 reactions that had produced a band of equal or superior size the 500 pairs of bases (bp), for one second round of analysis had been selected. The 28 selected mixtures of clones had been used as source of DNA for an amplification using the oligonucleotides T7 (for the present sequence in the vector pSPORT-P) and oligo 10. The result demonstrated the presence of bands, with the waited size (500 bp or more), in 8 of the analyzed mixtures. Three of these mixtures had been chosen for one third round of analysis, of this time through, the re-amplification of the fragments gotten in first and the second rounds, however with different oligonucleotides (oligos 11 and 12). The amplification from the 3 mixtures (position E07, G07 and F08) resulted in the appearance of a band of waited size and confirmed the presence of clones of DCNcA for KM+, in each one of the 3 mixtures.

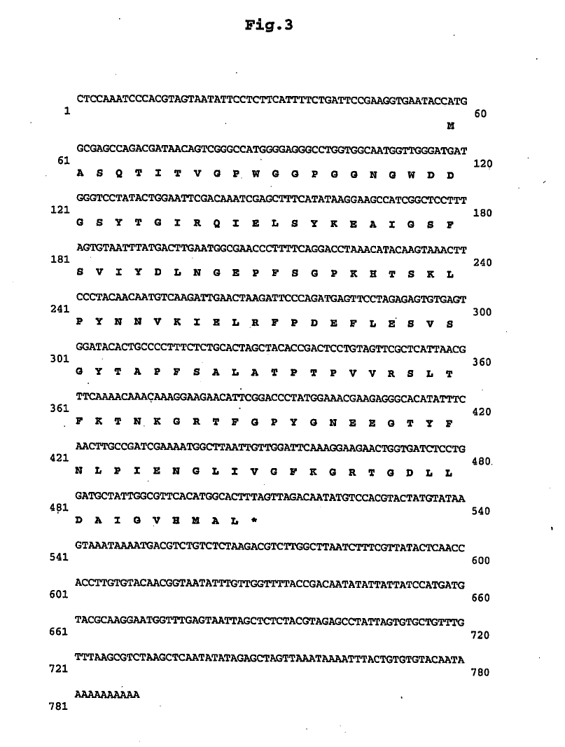

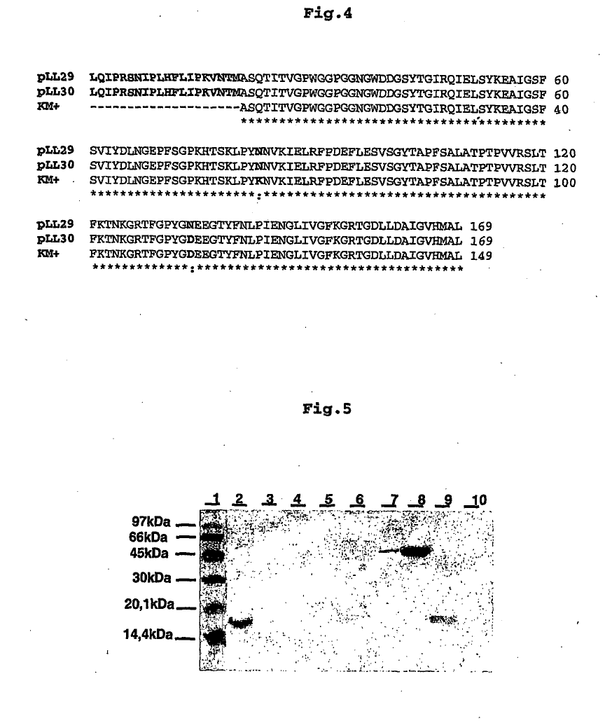

Of the 136 original plates, I contend only one clone of DCNcA in each well, had been removed aliquot of the supply in glicerol, that, in turn, they had served to initiate cultures in a new plate of 96 wells. In this stage, only clones in the positions E07 and F07 had been used (F08 was not analyzed to simplify the work). The 136 clones of the E07 position and the 136 clones of the F07 position had been reorganized in new plates that had again served as starting point for the assembly of a new matrix. This second matrix resulted of the mixture of all clones placed in one same line or one same column, in a total of 36 mixtures (FIG. 3, SEQ. ID NO:2). These mixtures had been used as source of DNA for amplification for PCR using the oligos T7 and 11. The result of the amplifications allowed to define the position of clones of DCNcA for KM+ (A* 12 or pLL30 and E*01 or pLL29), that they had been removed of the plates of the original library and had been used for the preparation of DNA and sequencing (FIG. 4, SEQ ID NO:3, SEQ. ID NO:4, SEQ. ID. NO5). FIG. 5 shows the alignment of 3 distinct amino acid sequences, corresponding the 3 isoforms of KM+ (one of them gotten through the sequencing of the protein and the others two, amino acid sequences deduced from the sequences of nucleotides of clones pLL29 and pLL30). Isoforms of the protein KM+, resultants of such forms of heterolog expression, anti-KM+, the selectivity of linking had kept the antigenicity front to the policlonal antibody the D-manose and to the trimanoside Man alfal-3 [Man alfal-6] Man, when tested front to a panel of sugars. In alive and in had been also capable to induce the migration of neutrophils vitro and to induce murines peritoneal cells to produce citocines of standard TH1.

### OBJECTS OF THE INVENTION

More advantageously the invention understands pharmaceutical composition for imunomodulation holding lectin KM+.

Still more preferential the invention understands pharmaceutical composition holding lectin KM+.

Still more advantageously the invention understands insecticidal composition holding lectin KM+.

Preferential the invention understands medicine holding lectin KM+.

Advantageously the invention understands insecticide holding lectin KM+.

Still more preferential the invention understands the use of lectin KM+ in the imodulator medicine preparation.

Advantageously the invention understands the use of lectin KM+ in the medicine preparation to prevent or to deal with decurrent injuries chemical or physical aggressions.

More advantageously the invention understands the use of lectin KM+ in the anti-bacterial medicine preparation.

More preferential the invention understands the use of lectin KM+ in the anti-viral medicine preparation.

Still more preferential the invention understands the use of lectin KM+ in the anti-parasitic medicine preparation.

Advantageously the invention understands the use of lectin KM+ in the anti-fungal medicine preparation.

Still more advantageously the invention understands the use of lectin KM+ in the preparation of insecticide.

Still more advantageously the invention understands the use of lectin KM+ in the preparation of insecticidal composition.

Preferential the invention understands the use of lectin KM+ for recognition of manose.

More preferential the invention understands the use of lectin KM+ for purification of proteins contends manose.

Still more preferential the invention concerns to the method of expression of lectin KM+ being effected from DCNcA or genomic sequence or synthetic sequence for the native form.

Advantageously the invention mentions to it method of expression of lectin KM+ being effected in organism from DCNcA or genomic sequence or synthetic sequence in fusing with other proteins and peptides.

More advantageously the method of expression of lectin KM+ can be effected in organism from DCNcA or genomic sequence or synthetic sequence in fusing with peptides.

Preferential the method of expression of lectin KM+ can be effected in organism from DCNcA or genomic sequence or synthetic sequence in fusing with part of the original protein.

Still more advantageously the expression method holds organism understanding procariote or eucariote. Preferential the expression method holds procariote understanding bacteria.

More preferential the expression method holds bacteria understanding Escherichia coli.

Still more preferential the expression method holds bacteria understanding Caulobacter.

Preferential the expression method understands bacteria infectated with recombinant virus.

Advantageously the expression method holds eucariote understanding Saccharomyces.

More advantageously the expression method holds Saccharomyces understanding S. cerevisiae.

Preferential the expression method holds Schizosaccharomyces understanding the S. pombe.

More preferential the expression method holds eucariote understanding Pichia.

Still more preferential the expression method holds Pichia understanding the P. pastoral.

Advantageously the expression method holds Pichia understanding the methanolica P.

More advantageously the expression method holds eucariote understanding plants.

Still more advantageously the expression method understanding transgenics plants.

Preferential the expression method understands plants infectated with recombinant virus.

More preferential the expression method understands eucariote holding vegetal cells. Still more preferential the expression method holds eucariote understanding animal.

Advantageously the expression method holds animals understanding transgenics.

More advantageously the expression method holds animals understanding mammals.

Preferential the expression method understands the job of mammal cells.

More preferential the expression method holds animals understanding insects.

Still more preferential the expression method holds eucariote understanding animal transgenics and cells of mammals.

Advantageously the expression method holds eucariote understanding cells of insects.

Preferential the method of expression of lectin in And coli, can be effected from DCNcA subclonado in the vector pDEST14.

More preferential the vector of relative DNA understands the SEQ. ID. NO: 1-5.

Advantageously the DNA vector understands relative SEQ. ID. NO: 1-5 P F S G PK.

Advantageously the DNA vector understands relative SEQ. ID. NO: 1-5 K L P Y KN.

Advantageously the DNA vector understands SEQ. ID. NO: 1-5 A I G V H M to.

Still more advantageously the vector I contend the gene of the lectin understands the SEQ. ID NO: 2.

Preferential the recombinant organism understands the SEQ. ID. NO: 1-5.

More preferential the recombinant organism contains part of this protein.

Advantageously the DNA sequence can make pareament with the SEQ. ID NO: 2, can pairing, SEQ. ID NO: 1-5.

Preferential the sequence of nucleotides can codify one of the amino acid sequences of lectin KM+.

More preferential the lectin protein of Artocarpus integrifolia mentions the SEQ. ID NO: 1-5.

These and other objects of the present invention will become apparent to those skilled in the art from a review from the description provided below.

### SUMMARY OF THE INVENTION

The present invention refers it pharmaceutical composition to heal or to prevent epithelial injuries understanding lectin KM+. Advantageously it understands the epithelial injuries holding the cutaneous injuries. Preferential the epithelial injuries understand the injuries of the cornea.

### BRIEF DESCRIPTION OF THE INVENTION

The following figures are part of the present application:

FIG. 1 is an alignment of the amino acid sequences of lectins KM+ and jacaline, SEQ. ID NO: 1.

FIG. 2 is an amino acid sequence of the regions of KM+ chosen for the construction of oligonucleotides.

FIG. 3 is a schematical drawing of the matrix planned, consisting of three plates, SEQ. ID NO: 2.

FIG. 4 is a sequence of nucleotides of the present DCNcA of KM+ in the plasmid deduced amino acid pLL29, SEQ. ID NO: 3, SEQ. ID NO: 4, SEQ. ID NO: 5.

FIG. 5 is an alignment of the amino acid sequences deduced from the DCNcAs of the plasmids pLL29 and pLL30 and the gotten one from chemical analyses of the protein and depositing in data base (Pink et al., 1999).

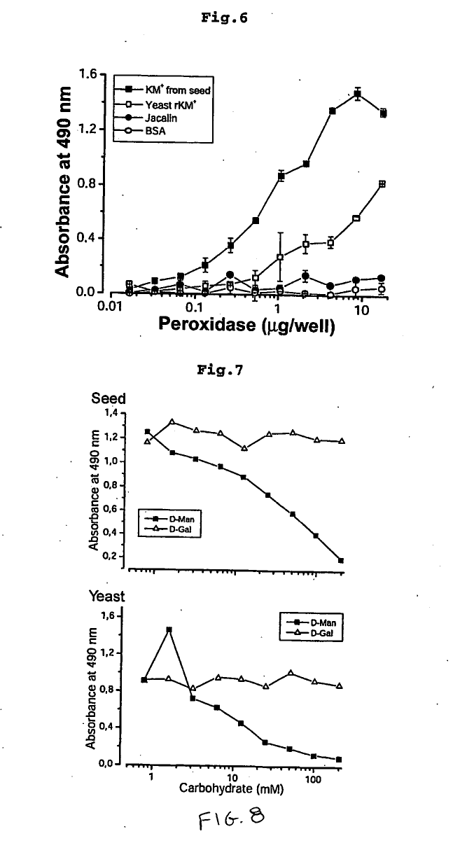

FIG. 6 is a graphical representation of a resultant membrane of the Western blot showing corresponding bands to the recombinant proteins KM+ and produced Gst-km+ in And coli, and protein KM+ produced in S. cerevisiae.

FIG. 7 is a graphical that compares the activity of linking with the glycoprotein (peroxidase) exerted by lectin KM+ derived from jaca seeds and by lectin KM+ gotten from the expression in heterologic system (lectin recombinant KM+).

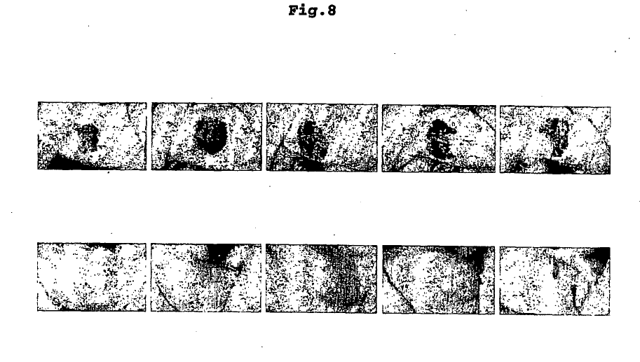

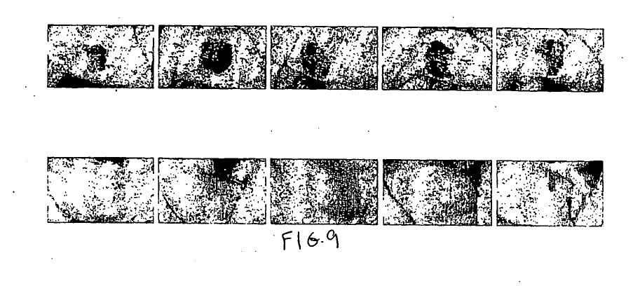

FIG. 8 is a graphical that shows the specific inhibition for monossacaride D-manose of the linking of KM+ of recombinant plant or KM+ to the glycoprotein (peroxidase).

FIG. 9 are photographs of decurrent injuries of burnings in the back of rats submitted to the topic application of lectin KM+ or only of the vehicle.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Relative ID to FIGS. 1 the 9. Advantageously the lectin protein of Artocarpus integrifolia mentions the Seqs to it. Relative ID to cited FIGS. 1 the 9 and any of isoforms. Preferential the lectin protein of Artocarpus integrifolia mentions the Seqs to it. Relative ID to cited FIGS. 1 the 9 and any of isoforms. More preferential the method of expression of lectin KM+ in S. cerevisiae, can be effected from DCNcA subclonado in the vector pYES-DEST52. Advantageously the plasmid of expression can contain as inserto the DNA sequence that codifies for lectin protein KM+ of Artocarpus integrifolia. More advantageously the plasmid of expression can use the relative information to the gene of lectin KM+ of Artocarpus integrifolia. Observing itself that lectin KM+, through the property to induce macrophages to produce I1-12 and to exert protective effect against infection for L. major and knowing itself that the brasiliensis resistance to the infection for Paracoccidioides depends on an efficient Th1 reply, evaluated the effect of the inoculation of KM+ in mice that would be defied with leavenings of P. brasiliensis. Groups of animals had been injected with KM+, associate or not its cell free antigen (CFA) proceeding from isolated Pb 18 of P. brasiliensis. Animals of another group had been injected with alone CFA and a group control received PBS.

The animals intravenously had been infectated with isolated leavenings of virulent of P. brasiliensis and, in the following period, monitored how much the diverse parameters, including the proliferactive reply of esplenic cells, recovery of formator units of colonies (CFU) in the fabric pulmonary and histopatologye of pulmonary injuries. The depression of proliferactive, proper reply of the infection, was reverted in the animals daily pay-inoculated with KM+. The recovery of formator units of colonies in the fabric pulmonary was carried through and was verified that the proportionate amount of colonies for the lungs of the animals daily pay-inoculated with KM+, was very inferior to the gotten one in the lungs of the animals that had not received the lectin. The pulmonary histopatologye showed that the mice daily pay-inoculated with KM+ showed size and significantly lesser number of granulomes how much to not treated. Such results indicate that KM+ intervenes positively with the course of the infection for P. brasiliensi, and that such imunomodulatory effect if makes independently of any process of immunization with fungic antigen. Lectin KM+ was incorporated in ointment on the basis of petroleum jelly, added or not of acid linoleic and also in formularization the base of propilenoglicol. The incorporation can be made in conventional dermatological bases (remarcably emulsions water in oil, hydrophilic and gel emulsions oil in water, ointment hidrofobics, ointment), as well as in elaborated systems of release more, contends or not promotional substances of the cutaneous absorption (remarcably propilenoglicol, acid oleic, derivatives of pirrolidona, etanol, dimetilsulfoxido, and fosfolipides) as multiple emulsions, micron and nanoemulsion or emulsions sucromicas, lipossomes, transferssomes, ethossomes and other types of vesicles, nanossomes, beyond other micron and nanoparticulados systems, as micron and nanocapsules, micron and nanoespheres and vehicles I contend ceramides, liquid, complex crystals of inclusion as that one with ciclodextrines, supersaturated solutions, formularizations with macro-molecules capable to form gel and films. To improve still more the penetration they could be gotten pro-pharmaceuticals of the substance (as ester remarcably), leaving it with a rocking hidro and adjusted lipophilic more for the penetration in the followed corneo stratum of conversion in the skin for the free pharmaceutical. All these pharmaceutical forms and systems of release of the pharmaceutical can be placed in devices that still more improve the performance of the substance for determined required action, as, remarcably, transdermic powderject (it shoots particles through the corneo stratum for the layers deepest of the skin for supersonic waves of gas shock helium) intraject (uses gas source compressed nitrogen to project doses of formularization through the skin for the fabric subcutaneous), microneedles, iontoforese, eletroporaction and high voltage (100 the 1000 V), sonoforese. The acute influx of inflammatory cells for KM+ based the hypothesis of that the lectin could have anti-microbian action to the being applied in injured cutaneous surfaces that, as well known, correspond the important door of entrance of patogens. The secondary infection corresponds the cause most frequent of death of patients with extensive losses of continuity of the cutaneous surface, as the ones that occur in great burnt. To test the hypothesis of, that the influx of neutrophils provoked by the lectin could diminish the infection incidence, rats had been submitted the thermal aggression in the dorsal region and topically treated with ointment I contend KM+.

Surprising the action of KM+ if it made to after notice since few hours the application of the ointment one, and it was not related with the combat to the secondary infections, but yes to a precocious regeneration of the fabric injured. The animals dealt with KM+ differentiated themselves of the not treat ones (to which the vehicle was applied only) for the absence of necro-hemorrhagic areas, or deep injuries that they displayed weaved muscular. In contrast regeneration if made quickly, the edges of the injuries was little salient, was come close quickly, having precocious cicatrizaction of the wound, manifest for local reepitelizaction and ressurgiment of pilificaction. Many experiments, of varied characteristics had been repeated, with superficial or deep injuries, different burnings of ample or small diameters, times of exposition to the heat, use of dry or humid heat. In all the experimental circumstances the animals if had benefited of the local application of KM+ In FIG. 9 are the photographs of the dorsal region of two groups of animals, 5 treated topically with lectin KM+ (3 applications), and treated others 5 only with the vehicle (3 applications). All had suffered to thermal injury in the back 24 hours before the photo. The rats dealt with KM+ (inferior photograph series) had had the fabric preserved, while the ones that had received only the vehicle (superior photograph series) present local necro-hemorrhagic injuries. The dramatically beneficial action of the application of KM+ in the fabrics is clear that had suffered thermal aggression.

1-63. (canceled)

64. A method of providing expression of a lectin in an organism, where the lectin is derived from cDNA, genomic sequence, or synthetic sequence in the native form.

65. A lectin expression method according to claim 64 wherein said expression is carried out with a sequence fused with other proteins and peptides.

66. A lectin expression method according to claim 64 wherein said expression is carried out with a sequence fused with peptides.

67. A lectin expression method according to claim 64 wherein said expression is carried out with a sequence fused with part of the original protein.

68. A lectin expression method according to any claims 64 wherein said lectin are the members of jacalin-related family.

69. A lectin expression method according to any claims 64 wherein said lectin are the D-mannose binding members of the jacalin-related family.

70. A lectin expression method according to any claims 64 wherein said lectin is KM+.

71. An expression method according to any claims 64 wherein the organism comprises a prokaryote or eukaryote.

72. An expression method according to claim 71, wherein the prokaryote comprises bacteria.

73. An expression method according to claim 72, wherein the bacteria comprises Escerichia coli.

74. An expression method according to claim 72, wherein the bacteria comprises Caulobacter.

75. An expression method according to any of claims 72, wherein the bacteria are infected with recombinant virus.

76. An expression method according to claim 71, wherein the eukaryotes comprise Saccharmices.

77. An expression method according to claim 76, wherein the Saccharomices comprises S. cerevisae.

78. An expression method according to claim 76, wherein the Saccharomices comprises S. pombe.

79. An expression method according to claim 71, wherein the eukaryote comprises Pichia.

80. An expression method according to claim 79, wherein the Pichia comprises P. pastoris.

81. An expression method according to claim 79, wherein the Pichia comprises P. methanolica.

82. An expression method according to claim 71, wherein the eukaryote comprises plants.

83. An expression method according to claim 82, wherein the plants are transgenic.

84. An expression method according to claim 82, wherein the plants are infected with recombinant virus.

85. An expression method according to claim 71, wherein the eukaryote comprises vegetable cells.

86. An expression method according to claim 71, wherein the eukaryote comprises animal cells.

87. An expression method according to claim 86, wherein the animals are transgenic.

88. An expression method according to claim 86, wherein the animals are mammals.

89. An expression method according to claim 86, wherein the mammal cells are used.

90. An expression method according to claim 86, where in the animals comprise insects.

91. An expression method according to claim 71, wherein the eukaryote comprises transgenic and mammal cells.

92. An expression method according to claim 71, wherein the eukaryote comprises insect cells.

93. A lectin expression method according to any claims 64 when the organism comprises a prokaryote and expression is carried out from subcloned cDNA in pDEST14 vector.

94. A lectin expression method according to claim 93, wherein the prokaryote comprises bacteria.

95. A lectin expression method according to claim 94, wherein the bacteria comprises Escherichia coli.

96. A lectin expression method according to claim 94, wherein the bacteria comprises Caulobacter.

97. A lectin expression method according to any claims 93, wherein the said lectin is KM+.

98. A DNA vector comprising any one of SEQ. ID NO: 1, 2, 3, 4 or 5.

99. A DNA vector according to claim 98, wherein the sequence is Glu Pro Phe Ser Gly Pro Lys.

100. A DNA vector according to claim 98, wherein the sequence is Pro Tyr Lys Asn.

101. A DNA vector according to claim 98 wherein the sequence is Ala Ile Gly Val His Met Ala.

102. A vector according to claim 98, wherein the sequence is Gly Val His Met Ala.

103. A vector containing the lectin gene comprising any of SEQ. ID NO: 1 through 5.

104. A recombinant organism comprising any of SEQ. ID NO: 1 through 5.

105. A recombinant organism according to claim 104 comprising the fact of containing the protein.

106. A DNA sequence comprised of a pairing off with any of SEQ. ID NO: 1 through 5.

107. A nucleotide sequence codifying at least one lectin amino acid sequence having any of SEQ. ID NO: 1 through 5.

108. A nucleotide sequence according to claim 107, wherein the lectin is KM+.

109. A lectin protein comprising any of SEQ. ID NO: 1 through 5.

110. A lectin protein according to claim 109, wherein said lectin protein is of Artocarpus integrifolia.

111. A lectin protein comprising any of SEQ. ID NO: 1 through 5.

112. A lectin protein according to claim 111, wherein said lectin protein is of Artocarpus integrfolia.

113. A lectin protein according to any claims 109 to 112 wherein the sequence is Glu Pro Phe Ser Gly Pro Lys.

114. A lectin protein according to any claims 109 to 112 wherein the sequence is Lys Leu Pro Tyr Lys Asn.

115. A lectin protein according to any claim 109 to 112 wherein the sequence is Ala Ile Gly Val His Met Ala.

116. A lectin expression method in a eukaryote or prokaryote carried out from subcloned cDNA in pYES-DEST52.

117. A lectin expression method according to claim 116, wherein said lectin is KM+.

118. A lectin expression method according to claims 116 or 117, wherein said prokaryote us bacteria.

119. A lectin expression method according to claim 118, wherein the said bacteria is S. cerevisae.

120. A vector expression plasmid comprising a DNA sequence that codifies for the lectin protein.

121. A vector expression plasmid of claim 120, wherein said lectin protein is KM+.

122. An expression plasmid of claims 120 or 121 comprising the lectin gene.

123. An expression plasmid of claim 122, wherein said lectin gene is KM+.

124. An expression plasmid of claim 120, wherein the said lectin KM+ gene is of Artocarpus integrifolia.

125. A pharmaceutical composition to treat epithelial wounds comprising lectin according to any claims 111 to 112.

126. A pharmaceutical composition of claim 125, wherein the epithelial wounds are skin wounds.

127. A pharmaceutical composition according to claim 126, wherein the skin wounds are cornea wounds.

128. A pharmaceutical composition for immunomodulating comprising lectin according to any claims 111 to 112.

129. An insecticide comprising lectin according to any claims 111 to 112.

130. The use of lectin according to any claims 111 to 112 comprising the preparation of medicine for treatment of wounds as a cicatrizer.

131. The use of lectin according to any claims 111 to 112 comprising the preparation of medicines to prevent wounds.

132. The use of lectin according to claims 127, wherein the wounds resulted from chemical or physical aggressions.

133. The use of lectin according to any claims 111 to 112 comprising the preparation of anti-bacterial medicine.

134. The use of lectin according to any claims 111 to 112 comprising the preparation of antiviral medicines.

135. The use of lectin according to any claims 111 to 112 comprising the preparation of anti-parasite medicines.

136. The use of lectin according to any claims 111 to 112 comprising the preparation of anti-fungal medicine.

137. The use of lectin according to any claims 111 to 112 comprising the preparation of insecticide.

138. The use of lectin according to any claims 111 to 112 comprising the preparation of mannose.

139. The use of lectin according to any claims 111 to 112 comprising the purification or detection of protein containing mannose.


SEQUENCE LISTING (5 sequences)

SEQ ID NO: 1 - protein, 302 aa, Artocarpus integrifolia
ASQTITVGPWGGPGGNGWDDGSYTGIRQIELSYKEAIGSFSVIYDLNGEPFSGPKHTSKLPYKNVKIELRFPDEFIESVSGYTAPFSALATPTPVVRSLTFKTNKGRTFGPYGDEEGTYFNLPIENGLIVGFKGRTGDLLDAIGVHMALLSLYMSFYDLWYGISGKFGVILGNEIPLNFPTGSTVGYPGYTKKNTKFTLSRVVVYGSVNGTYGSVEMIYESPFDLSIKVPTFGTIFSKHNQGVYPSGNLDYVVQFDGIATEKNYSLNIERIGTFAGDDFAKGSVKAGWSGVIVTQSKGSQEN

SEQ ID NO: 2 - DNA, 790 bp, Artocarpus integrifolia
ctccaaatcccacgtagtaatattcctcttcattttctgattccgaaggtgaataccatggcgagccagacgataacagtcgggccatggggagggcctggtggcaatggttgggatgatgggtcctatactggaattcgacaaatcgagctttcatataaggaagccatcggctcctttagtgtaatttatgacttgaatggcgaacccttttcaggacctaaacatacaagtaaacttccctacaacaatgtcaagattgaactaagattcccagatgagttcctagagagtgtgagtggatacactgcccctttctctgcactagctacaccgactcctgtagttcgctcattaacgttcaaaacaaacaaaggaagaacattcggaccctatggaaacgaagagggcacatatttcaacttgccgatcgaaaatggcttaattgttggattcaaaggaagaactggtgatctcctggatgctattggcgttcacatggcactttagttagacaatatgtccacgtactatgtataagtaaataaaatgacgtctgtctctaagacgtcttggcttaatctttcgttatactcaaccaccttgtgtacaacggtaatatttgttggttttaccgacaatatattattatccatgatgtacgcaaggaatggtttgagtaattagctctctacgtagagcctattagtgtgctgtttgtttaagcgtctaagctcaatatatagagctagttaaataaaatttactgtgtgtacaataaaaaaaaaaa

SEQ ID NO: 3 - protein, 169 aa, Artocarpus integrifolia
LQIPRSNIPLHFLIPKVNTMASQTITVGPWGGPGGNGWDDGSYTGIRQIELSYKEAIGSFSVIYDLNGEPFSGPKHTSKLPYNNVKIELRFPDEFLESVSGYTAPFSALATPTPVVRSLTFKTNKGRTFGPYGNEEGTYFNLPIENGLIVGFKGRTGDLLDAIGVHMAL

SEQ ID NO: 4 - protein, 169 aa, Artocarpus integrifolia; identical to SEQ ID NO: 3

SEQ ID NO: 5 - protein, 149 aa, Artocarpus integrifolia
ASQTITVGPWGGPGGNGWDDGSYTGIRQIELSYKEAIGSFSVIYDLNGEPFSGPKHTSKLPYNNVKIELRFPDEFLESVSGYTAPFSALATPTPVVRSLTFKTNKGRTFGPYGNEEGTYFNLPIENGLIVGFKGRTGDLLDAIGVHMAL